(19) United States
(12) Patent Application Publication
(10) Pub. No.: US 2025/0352371 A1
(43) Pub. Date: Nov. 20, 2025

(54) ENDOPROSTHESIS WITH IMPROVED CHARACTERISTICS

(71) Applicant: BOSTON SCIENTIFIC SCIMED, INC., Maple Grove, MN (US)

(72) Inventors: GARY GILMARTIN, Foxford (IE); DANIEL TUCK, Galway (IE); CELINE GLYNN, Galway (IE); FIONN STAPLETON, Galway (IE); JOHN THOMAS O'DRISCOLL, Galway (IE); THOMAS MARTIN KEATING, Galway (IE); HARRY MULLEADY, Galway (IE)

(73) Assignee: BOSTON SCIENTIFIC SCIMED, INC., Maple Grove, MN (US)

(21) Appl. No.: 19/208,807

(22) Filed: May 15, 2025

Related U.S. Application Data
(60) Provisional application No. 63/648,428, filed on May 16, 2024.

(51) Int. Cl. A61F 2/89

(52) U.S. Cl. CPC A61F 2/89; A61F 2220/0075

(57) ABSTRACT

An endoprosthesis includes a body portion extending from a first end to a second end along a central longitudinal axis. The body portion is formed from a single wire extending from the first end to the second end and forming a plurality of circumferential segments extending non-helically around the axis. The plurality of circumferential segments includes a first end circumferential segment forming the first end, a second end circumferential segment forming the second end, and at least one medial circumferential segment disposed between the first and second end circumferential segments. Each circumferential segment includes an undulating arrangement of first struts and second struts defining peaks and valleys, the first and second struts being disposed at first and second angles, respectively, relative to the axis in a side view of the body portion. Peaks and valleys of adjacent circumferential segments may be axially aligned with each other parallel to the axis.

### CROSS-REFERENCE TO RELATED APPLICATIONS

The application claims the benefit of U.S. Provisional Patent Application Ser. No. 63/648,428, filed on May 16, 2024, the disclosure of which is incorporated herein by reference.

### TECHNICAL FIELD

The present disclosure pertains to medical devices, methods for manufacturing medical devices, and uses thereof. More particularly, the present disclosure pertains to an endoprosthesis or stent for implantation in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, and bile duct, as well as in a variety of other applications in the body.

In some instances, it may be desirable to design an endoprosthesis to include sufficient flexibility and conformability to the body lumen, while maintaining sufficient radial force to open the body lumen at the treatment site and/or prevent migration of the endoprosthesis within the body lumen. In some instances, it may be desirable to reduce or limit foreshortening. In some instances, different endoprosthesis configurations may provide different deliverability, flexibility, conformability, radial force/strength, and/or anchoring/migration characteristics.

Of the known medical devices and methods, each has certain advantages and disadvantages. There is an ongoing need to provide alternative medical devices as well as alternative methods for manufacturing and using medical devices.

### SUMMARY

In one example, an endoprosthesis may comprise a body portion extending axially from a first end to a second end along a central longitudinal axis. The body portion may be formed from a single wire extending from the first end to the second end. The single wire may form a plurality of circumferential segments extending non-helically around the central longitudinal axis.

In addition, or alternatively, to any example disclosed herein, each circumferential segment of the plurality of circumferential segments comprises an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion.

In addition, or alternatively, to any example disclosed herein, each circumferential segment of the plurality of circumferential segments is connected to an axially adjacent circumferential segment of the plurality of circumferential segments by a transition segment of the single wire disposed at a third angle relative to the central longitudinal axis in the side view of the body portion, wherein the third angle is different from the first angle and the second angle.

In addition, or alternatively, to any example disclosed herein, all transition segments of the single wire collectively define a transition zone.

In addition, or alternatively, to any example disclosed herein, the transition zone extends parallel to the central longitudinal axis.

In addition, or alternatively, to any example disclosed herein, the transition zone extends helically around the central longitudinal axis.

In addition, or alternatively, to any example disclosed herein, the endoprosthesis may comprise a polymeric covering fixedly attached to the body portion.

In addition, or alternatively, to any example disclosed herein, the body portion is embedded within the polymeric covering.

In addition, or alternatively, to any example disclosed herein, at least one circumferential segment of the plurality of circumferential segments comprises an anti-migration element projecting radially outward from the at least one circumferential segment, wherein the anti-migration element is formed from the single wire.

In addition, or alternatively, to any example disclosed herein, and in a second example, an endoprosthesis may comprise a body portion extending axially from a first end to a second end along a central longitudinal axis. The body portion may be formed from a single wire extending continuously from the first end to the second end. The single wire may form a plurality of circumferential segments extending non-helically around the central longitudinal axis. The plurality of circumferential segments may comprise a first end circumferential segment forming the first end, a second end circumferential segment forming the second end, and at least one medial circumferential segment disposed axially between the first end circumferential segment and the second end circumferential segment.

In addition, or alternatively, to any example disclosed herein, each circumferential segment of the plurality of circumferential segments comprises an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion.

In addition, or alternatively, to any example disclosed herein, each circumferential segment of the plurality of circumferential segments is connected to an axially adjacent circumferential segment by a transition segment of the single wire disposed nonparallel to the central longitudinal axis, the first struts, and the second struts in the side view of the body portion.

In addition, or alternatively, to any example disclosed herein, the peaks and valleys of each circumferential segment define a circumferential segment height measured parallel to the central longitudinal axis, and the circumferential segment height is uniform among the plurality of circumferential segments.

In addition, or alternatively, to any example disclosed herein, the peaks and valleys of each circumferential segment define a circumferential segment height measured parallel to the central longitudinal axis, and the circumferential segment height varies among the plurality of circumferential segments.

In addition, or alternatively, to any example disclosed herein, a length of the body portion from the first end to the second end is configured to change by less than 5% when shifting between a collapsed delivery configuration and an expanded deployed configuration.

In addition, or alternatively, to any example disclosed herein, each adjacent pair of circumferential segments defines an axial spacing, and the axial spacing between adjacent pairs of circumferential segments is uniform along the body portion.

In addition, or alternatively, to any example disclosed herein, each adjacent pair of circumferential segments defines an axial spacing, and the axial spacing between adjacent pairs of circumferential segments varies along the body portion.

In addition, or alternatively, to any example disclosed herein, and in a third example, an endoprosthesis may comprise a body portion extending axially from a first end to a second end along a central longitudinal axis. The body portion may be formed from a single wire extending from the first end to the second end. The single wire may form a plurality of circumferential segments extending non-helically around the central longitudinal axis. Each circumferential segment of the plurality of circumferential segments may comprise an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion. The peaks and valleys of adjacent circumferential segments of the plurality of circumferential segments may be axially aligned with each other parallel to the central longitudinal axis.

In addition, or alternatively, to any example disclosed herein, each circumferential segment of the plurality of circumferential segments is connected to an axially adjacent circumferential segment of the plurality of circumferential segments by a transition segment of the single wire.

In addition, or alternatively, to any example disclosed herein, the transition segment extends between a valley of one circumferential segment and a peak of an immediately adjacent circumferential segment.

In addition, or alternatively, to any example disclosed herein, the transition segment is nonparallel with the first struts and second struts of the one circumferential segment and the first struts and second struts of the immediately adjacent circumferential segment in the side view of the body portion.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a flat pattern view illustrating selected aspects of an endoprosthesis in accordance with the disclosure;

FIG. 2 is a flat pattern view illustrating selected aspects of an endoprosthesis in accordance with the disclosure;

FIG. 3 is a flat pattern view illustrating selected aspects of an endoprosthesis in accordance with the disclosure;

FIG. 4 is a flat pattern view illustrating selected aspects of an endoprosthesis in accordance with the disclosure;

FIG. 5 is a flat pattern view illustrating selected aspects of an endoprosthesis in accordance with the disclosure;

FIG. 6 is a flat pattern view illustrating selected aspects of an endoprosthesis in accordance with the disclosure;

FIG. 7 illustrates selected aspects of an endoprosthesis in accordance with the disclosure using side and end views; and

FIGS. 8A-8B schematically illustrate selected aspects of an endoprosthesis system in accordance with the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

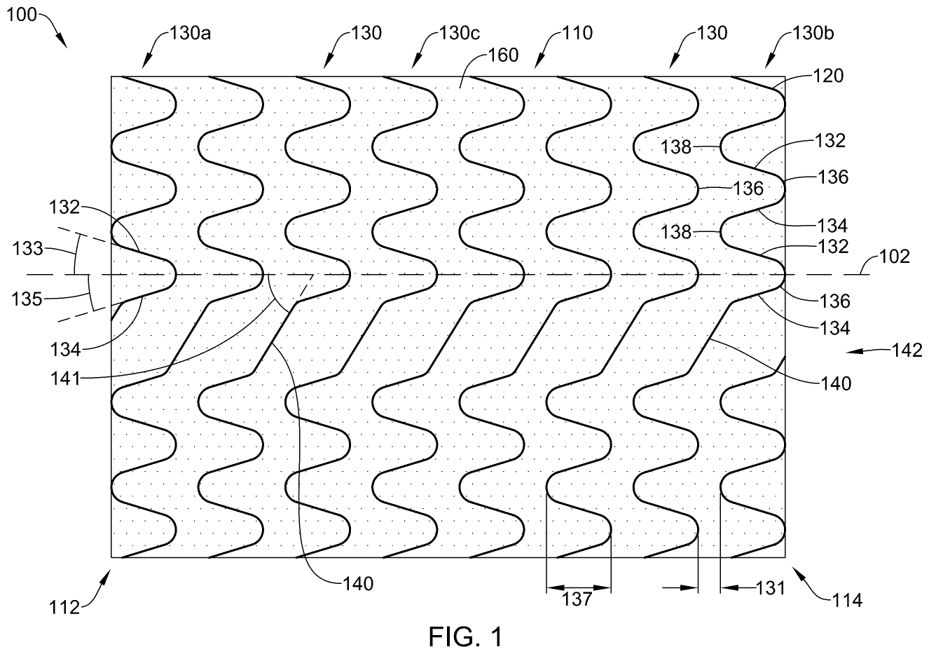

FIGS. 1-7 illustrate selected aspects of an endoprosthesis 100, such as a stent, wherein FIGS. 1-6 illustrate the endoprosthesis 100 in a flat pattern view. The endoprosthesis 100 may comprise a body portion 110 extending axially from a first end 112 to a second end 114 along a central longitudinal axis 102. In some embodiments, the first end 112 and the second end 114 may define a length of the endoprosthesis 100 and/or the body portion 110. In at least some embodiments, the body portion 110 may be tubular. The body portion 110 may define a lumen extending therethrough from the first end 112 to the second end 114.

In some embodiments, the endoprosthesis 100 may comprise a flared first end portion (not shown) and/or a flared second end portion (not shown), wherein an outer diameter of the flared first end portion is greater than an outer diameter of the body portion 110 and/or an outer diameter of the flared second end portion is greater than the outer diameter of the body portion 110. Other configurations are also contemplated. The flared first end portion may be disposed proximate and/or adjacent the first end 112. In some embodiments, the flared first end portion may extend from the first end 112 to the body portion 110. The flared second end portion may be disposed proximate and/or adjacent the second end 114. In some embodiments, the flared second end portion may extend from the second end 114 to the body portion 110.

In some embodiments, the outer diameter of the body portion 110 may be generally uniform and/or generally constant along the length of the endoprosthesis 100 and/or the body portion 110. In some embodiments, the outer diameter of the body portion 110 may be generally uniform and/or generally constant along the length of the body portion 110 except for the flared first end portion and/or the flared second end portion, where present. In some embodiments, the outer diameter of the body portion 110 may be generally uniform and/or generally constant along the length of the body portion 110 from the flared first end portion to the flared second end portion, where present. In some embodiments, the outer diameter of the body portion 110 may be between about 4 millimeters and about 40 millimeters, between about 6 millimeters and about 35 millimeters, between about 8 millimeters and about 30 millimeters, between about 10 millimeters and about 25 millimeters, etc. Other configurations are also contemplated.

In some embodiments, the endoprosthesis 100 and/or the body portion 110 may be formed from a single wire 120 extending from the first end 112 to the second end 114. In some embodiments, the flared first end portion and/or the flared second end portion, where present, may be formed from the single wire 120. The single wire 120 may extend circumferentially around the central longitudinal axis 102 and/or the single wire 120 may encircle the central longitudinal axis 102. The single wire 120 is and/or consists of only one wire.

In some embodiments, the single wire 120 may form a plurality of circumferential segments 130 extending non-helically around the central longitudinal axis 102. In some embodiments, the plurality of circumferential segments 130 may be substantially parallel to each other. In some embodiments, each circumferential segment of the plurality of circumferential segments 130 may form a ring extending around the central longitudinal axis 102. Some suitable but non-limiting examples of materials for the endoprosthesis 100 and/or the single wire 120, such as metallic materials, composite materials, shape memory materials, combinations thereof, etc., are discussed below. In one non-limiting example, the endoprosthesis 100 and/or the single wire 120 may be formed from nickel-titanium alloy (e.g., nitinol). In another non-limiting example, the endoprosthesis 100 and/or the single wire 120 may be formed from nickel-titanium alloy with a platinum core. Other configurations and/or materials are also contemplated.

In some embodiments, the plurality of circumferential segments 130 may comprise a first end circumferential segment 130A forming the first end 112, a second end circumferential segment 130B forming the second end 114, and at least one medial circumferential segment 130C disposed axially between the first end circumferential segment 130A and the second end circumferential segment 130B. In some embodiments, the at least one medial circumferential segment 130C may comprise one circumferential segment, two circumferential segments, three circumferential segments, etc. up to a desired number of circumferential segments producing a desired overall length for the endoprosthesis 100.

In some embodiments, each circumferential segment of the plurality of circumferential segments 130 may comprise an undulating arrangement (e.g., a sinusoidal arrangement) of first struts 132 and second struts 134 defining peaks 136 and valleys 138. In at least some embodiments, within any particular circumferential segment of the plurality of circumferential segments 130, the first struts 132 have a first length defined by the peaks 136 and the valleys 138 and the second struts 134 have a second length defined by the peaks 136 and the valleys 138, wherein the first length is equal to the second length. In some embodiments, the first length and the second length may be uniform and/or constant among the plurality of circumferential segments 130. In some alternative embodiments, the first length and the second length may vary among the plurality of circumferential segments 130. In some embodiments, the first length and/or the second length may be between about 1 millimeter and about 15 millimeters. In some embodiments, the first length and/or the second length may be between about 3 millimeters and about 12 millimeters. In some embodiments, the first length and/or the second length may be between about 5 millimeters and about 10 millimeters. Other configurations are also contemplated.

In some embodiments, the first struts 132 may be disposed at a first angle 133 relative to the central longitudinal axis 102 in a side view and/or a flat pattern view of the body portion 110 and/or the endoprosthesis 100, and the second struts 134 may be disposed at a second angle 135 relative to the central longitudinal axis 102 in the side view and/or the flat pattern view of the body portion 110 and/or the endoprosthesis 100. In at least some embodiments, the second angle may be different from the first angle. In some embodiments, the first angle 133 and/or the second angle 135 may be between zero degrees and 90 degrees. In some embodiments, the first angle 133 and/or the second angle 135 may be between 5 degrees and 60 degrees. In some embodiments, the first angle 133 and/or the second angle 135 may be between 10 degrees and 45 degrees. In some embodiments, the first angle 133 and/or the second angle 135 may be between 15 degrees and 30 degrees. Other configurations are also contemplated.

In some embodiments, the peaks 136 and the valleys 138 of adjacent circumferential segments of the plurality of circumferential segments 130 may be aligned with each other axially along the length of the body portion 110 and/or the endoprosthesis 100. For example, the peaks 136 of one circumferential segment of the plurality of circumferential segments 130 may be aligned axially with the peaks 136 of an immediately adjacent circumferential segment of the plurality of circumferential segments 130. Similarly, the valleys 138 of one circumferential segment of the plurality of circumferential segments 130 may be aligned axially with the valleys 138 of an immediately adjacent circumferential segment of the plurality of circumferential segments 130. In some embodiments, the peaks 136 and the valleys 138 of adjacent circumferential segments of the plurality of circumferential segments 130 may be aligned parallel to the central longitudinal axis 102. Other configurations are also contemplated.

In some embodiments, the peaks 136 and the valleys 138 of each circumferential segment of the plurality of circumferential segments 130 may be aligned with each other axially along the length of the body portion 110 and/or the endoprosthesis 100. For example, the peaks 136 of each circumferential segment of the plurality of circumferential segments 130 may be aligned axially with the peaks 136 of each other circumferential segment of the plurality of circumferential segments 130. Similarly, the valleys 138 of each circumferential segment of the plurality of circumferential segments 130 may be aligned axially with the valleys 138 of each other circumferential segment of the plurality of circumferential segments 130. In some embodiments, the peaks 136 and the valleys 138 of each circumferential segment of the plurality of circumferential segments 130 may be aligned parallel to the central longitudinal axis 102. Other configurations are also contemplated.

In some embodiments, each adjacent pair of circumferential segments of the plurality of circumferential segments 130 defines an axial spacing 131 therebetween. The axial spacing 131 may be measured axially and/or parallel to the central longitudinal axis 102. In some embodiments, the axial spacing 131 may be defined between the valleys 138 of one circumferential segment and the peaks 136 of an immediately adjacent circumferential segment. For the purpose of this disclosure, the axial spacing 131 shall be understood to not include any axial overlap with the first struts 132 and/or the second struts 134 of any given circumferential segment.

In some embodiments, the axial spacing 131 between adjacent pairs of circumferential segments of the plurality of circumferential segments 130 may be uniform along the length of the body portion 110 and/or the endoprosthesis 100, as seen in FIG. 1. In some embodiments, adjacent pairs of circumferential segments of the plurality of circumferential segments 130 are equally and/or uniformly spaced apart along the length of the body portion 110 and/or the endoprosthesis 100.

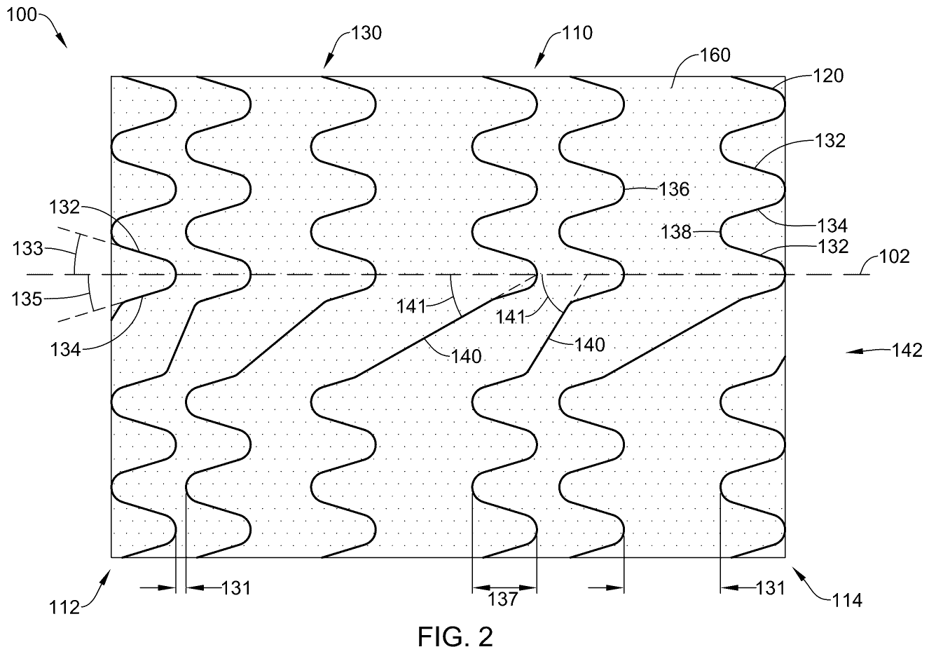

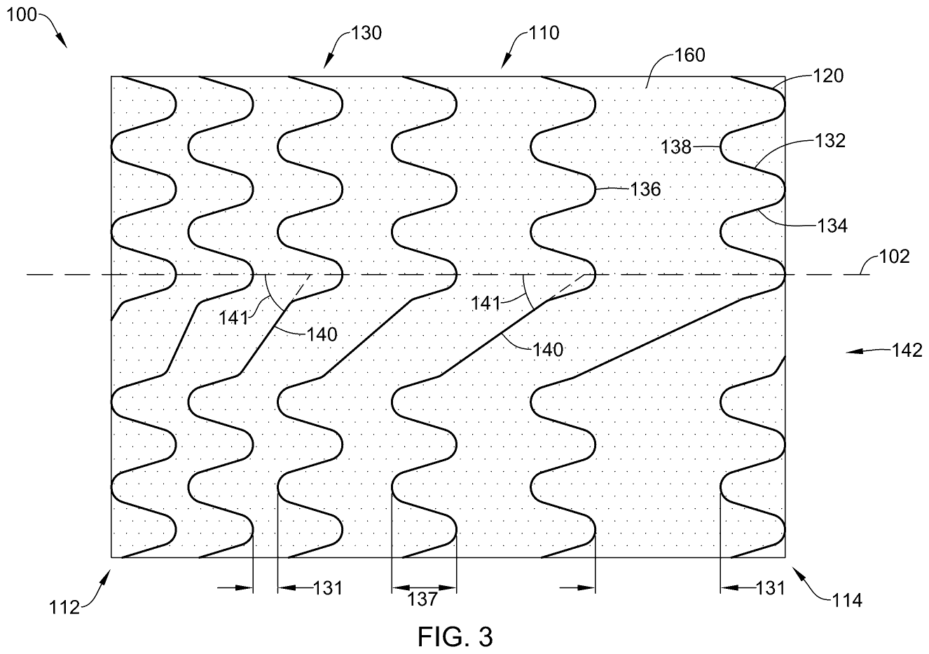

In some embodiments, the axial spacing 131 between adjacent pairs of circumferential segments of the plurality of circumferential segments 130 may vary along the length of the body portion 110 and/or the endoprosthesis 100, as seen in FIG. 2. In some embodiments, adjacent pairs of circumferential segments of the plurality of circumferential segments 130 are unequally and/or non-uniformly spaced apart along the length of the body portion 110 and/or the endoprosthesis 100. In some embodiments, the axial spacing 131 between adjacent pairs of circumferential segments of the plurality of circumferential segments 130 may increase along the length of the body portion 110 and/or the endoprosthesis 100 from the first end 112 toward and/or to the second end 114, as seen in FIG. 3. Alternatively, in some embodiments, the axial spacing 131 between adjacent pairs of circumferential segments of the plurality of circumferential segments 130 may decrease along the length of the body portion 110 and/or the endoprosthesis 100 from the first end 112 toward and/or to the second end 114.

In some embodiments, the peaks 136 and the valleys 138 of each circumferential segment of the plurality of circumferential segments 130 may define a circumferential segment height 137 for that circumferential segment measured parallel to the central longitudinal axis 102. In some embodiments, the circumferential segment height 137 may be uniform along the length of the body portion 110 and/or the endoprosthesis 100 and/or among the plurality of circumferential segments 130, as seen in FIG. 1.

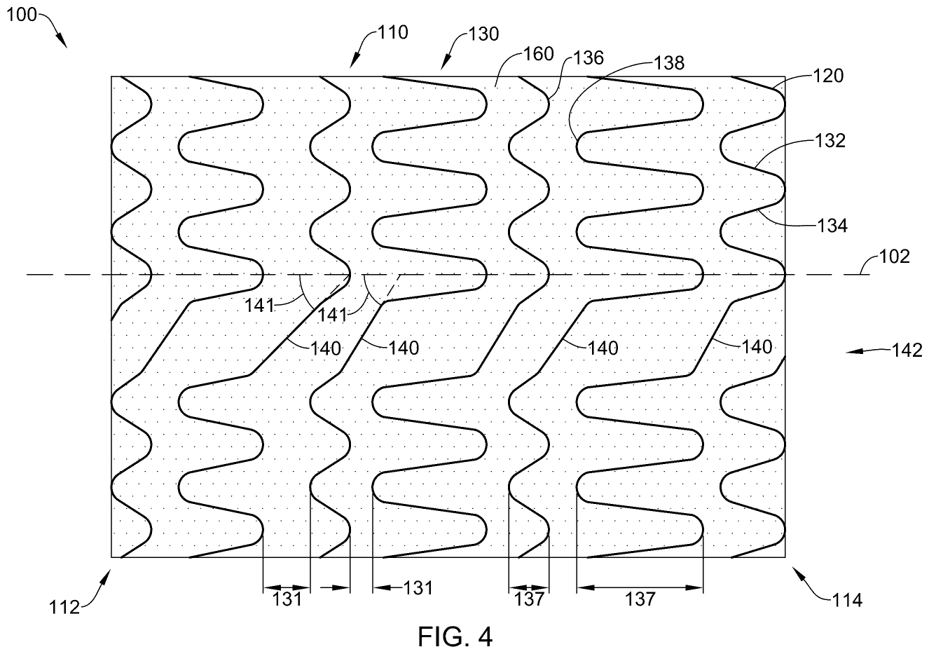

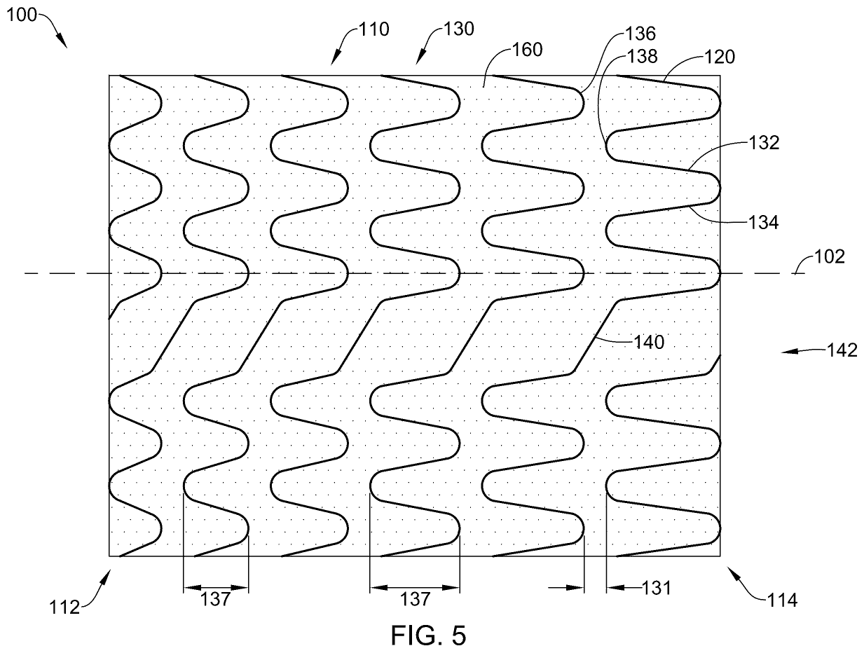

In some embodiments, the circumferential segment height 137 may vary along the length of the body portion 110 and/or the endoprosthesis 100 and/or among the plurality of circumferential segments 130, as seen in FIG. 4. In some embodiments, the circumferential segment height 137 may increase along the length of the body portion 110 and/or the endoprosthesis 100 and/or among the plurality of circumferential segments 130 from the first end 112 toward and/or to the second end 114, as seen in FIG. 5. Alternatively, in some embodiments, the circumferential segment height 137 may decrease along the length of the body portion 110 and/or the endoprosthesis 100 and/or among the plurality of circumferential segments 130 from the first end 112 toward and/or to the second end 114.

Returning to FIG. 1, in some embodiments, each circumferential segment of the plurality of circumferential segments 130 may be connected to an axially adjacent circumferential segment of the plurality of circumferential segments 130 by a transition segment 140 of the single wire 120. In at least some embodiments, each circumferential segment of the plurality of circumferential segments 130 may be discontinuous and/or may extend around less than a full circumference of the endoprosthesis 100 and/or the body portion 110. Instead, each circumferential segment of the plurality of circumferential segments 130 may have a first end and a second end, wherein the transition segment 140 extends between a first end of one circumferential segment of the plurality of circumferential segments 130 and a second end of an immediately adjacent circumferential segment of the plurality of circumferential segments 130 (or the transition segment 140 extends between a second end of one circumferential segment of the plurality of circumferential segments 130 and a first end of an immediately adjacent circumferential segment of the plurality of circumferential segments 130). In some embodiments, at least a portion of the first end circumferential segment 130A and/or the second end circumferential segment 130B may extend completely around the full circumference of the endoprosthesis 100 and/or the body portion 110, and the at least one medial circumferential segment 130C may be discontinuous and/or may extend around less than the full circumference of the endoprosthesis 100 and/or the body portion 110.

The transition segment 140 of the single wire 120 may be disposed at a third angle 141 relative to the central longitudinal axis 102 in the side view and/or the flat pattern view of the body portion 110 and/or the endoprosthesis 100. In at least some embodiments, the third angle 141 may be different from the first angle 133 and the second angle 135. In some embodiments, the transition segment 140 may have a length between about 5 millimeters and about 20 millimeters. In some embodiments, the transition segment 140 may have a length between about 8 millimeters and about 17 millimeters. In some embodiments, the transition segment 140 may have a length between about 10 millimeters and about 15 millimeters. Other configurations are also contemplated. In some embodiments, the third angle 141 may be between about 30 degrees and about 90 degrees. In some embodiments, the third angle 141 may be between about 35 degrees and about 75 degrees. In some embodiments, the third angle 141 may be between about 40 degrees and about 60 degrees. Other configurations are also contemplated.

In some embodiments, each circumferential segment of the plurality of circumferential segments 130 may be connected to an axially adjacent circumferential segment of the plurality of circumferential segments 130 by a transition segment 140 of the single wire 120 disposed nonparallel to the central longitudinal axis 102, the first struts 132, and the second struts 134 in the side view and/or the flat pattern view of the body portion 110 and/or the endoprosthesis 100. Other configurations are also contemplated.

In some embodiments, the transition segment 140 extending and/or disposed between axially adjacent circumferential segments of the plurality of circumferential segments 130 may extend between a valley (ref. 138) of one circumferential segment and a peak (ref. 136) of an immediately axially adjacent circumferential segment (or between a peak of one circumferential segment and a valley of an immediately axially adjacent circumferential segment). As illustrated, the transition segment(s) 140 extend from a peak (ref. 136) of a first circumferential segment disposed closer to the first end 112 to a valley (ref. 138) of a second circumferential segment immediately axially adjacent the first circumferential segment that is disposed closer to the second end 114 than the first circumferential segment. Other configurations are also contemplated. In some embodiments, the transition segment 140 may be disposed nonparallel with the first struts 132 and the second struts 134 of one circumferential segment (e.g., the first circumferential segment) and the first struts 132 and the second struts 134 of the immediately axially adjacent circumferential segment (e.g., the second circumferential segment).

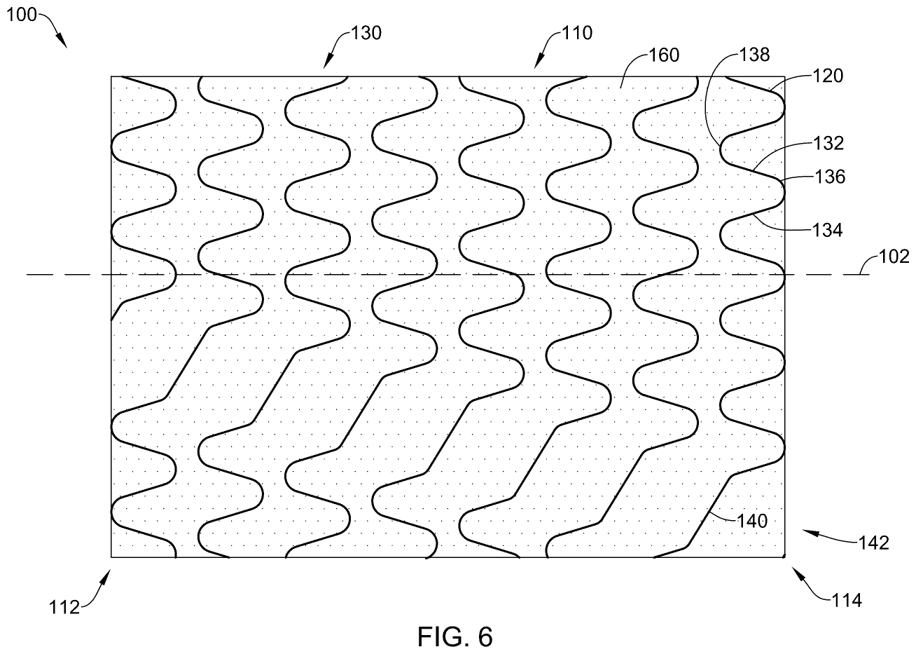

In some embodiments, all transition segments (e.g., an array of transition segments consisting of each transition segment 140 connecting and/or extending between adjacent circumferential segments of the plurality of circumferential segments 130) of the single wire 120 may collectively define a transition zone 142. In some embodiments, the transition zone 142 may extend parallel to the central longitudinal axis 102, as seen in FIG. 1. In some embodiments, the third angle 141 may be uniform along and/or within the transition zone 142. In some embodiments, each transition segment 140 of the single wire 120 may be aligned axially along the length of the body portion 110 and/or the endoprosthesis 100. In some embodiments, the transition zone 142 may define a preferential bending plane for the endoprosthesis 100 and/or the body portion 110 (e.g., the endoprosthesis 100 and/or the body portion 110 may be configured to bend more easily along and/or within the transition zone 142 than in other directions). In some embodiments, the third angle 141 may vary along and/or within the transition zone 142, as seen in FIGS. 2-3 for example. In some embodiments, the transition zone 142 may extend helically along and/or around the central longitudinal axis 102, as seen in FIG. 6.

In some embodiments, the peaks 136 and the valleys 138 of adjacent circumferential segments of the plurality of circumferential segments 130 may extend helically along the length of the body portion 110 and/or the endoprosthesis 100. In some embodiments, the peaks 136 and the valleys 138 of adjacent circumferential segments of the plurality of circumferential segments 130 may extend helically along and/or around the central longitudinal axis 102. In some embodiments, the peaks 136 and the valleys 138 of each circumferential segment of the plurality of circumferential segments 130 may extend helically along the length of the body portion 110 and/or the endoprosthesis 100. In some embodiments, the peaks 136 and the valleys 138 of each circumferential segment of the plurality of circumferential segments 130 may extend helically along and/or around the central longitudinal axis 102. Other configurations are also contemplated.

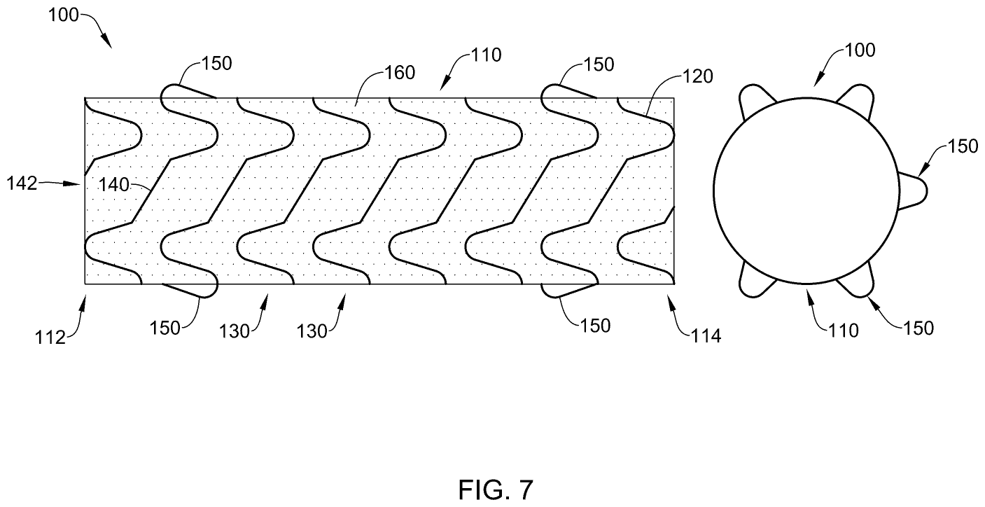

In some embodiments, the endoprosthesis 100 and/or the body portion 110 may comprise one or more anti-migration elements disposed along the length of the endoprosthesis 100 and/or the body portion 110. In some embodiments, at least one circumferential segment of the plurality of circumferential segments 130 may comprise an anti-migration element 150 projecting radially outward from the at least one circumferential segment and/or an outer surface of the endoprosthesis 100 and/or the body portion 110, as seen in FIG. 7. In some embodiments, at least one circumferential segment of the plurality of circumferential segments 130 may comprise a plurality of anti-migration elements projecting radially outward from the at least one circumferential segment and/or the outer surface of the endoprosthesis 100 and/or the body portion 110.

In some embodiments, anti-migration element(s) positioned proximate the first end 112 may project radially outward and toward the second end 114 (e.g., at a non-zero angle relative to the outer surface of the endoprosthesis 100 and/or the body portion 110) and/or anti-migration element(s) positioned proximate the second end 114 may project radially outward and toward the first end 112 (e.g., at a non-zero angle relative to the outer surface of the endoprosthesis 100 and/or the body portion 110). In some embodiments, anti-migration element(s) positioned proximate the first end 112 may project radially outward and toward the first end 112 (e.g., at a non-zero angle relative to the outer surface of the endoprosthesis 100 and/or the body portion 110) and/or anti-migration element(s) positioned proximate the second end 114 may project radially outward and toward the second end 114 (e.g., at a non-zero angle relative to the outer surface of the endoprosthesis 100 and/or the body portion 110). In some embodiments, some anti-migration elements positioned proximate the first end 112 may project toward the first end 112 and some may project the second end 114 (e.g., at a non-zero angle relative to the outer surface of the endoprosthesis 100 and/or the body portion 110), and/or some anti-migration elements positioned proximate the second end 114 may project toward the first end 112 and some may project the second end 114 (e.g., at a non-zero angle relative to the outer surface of the endoprosthesis 100 and/or the body portion 110). Other configurations are also contemplated.

In some embodiments, the endoprosthesis 100 may comprise a polymeric covering 160, shown in the figures via dotted shading, coupled to the body portion 110, the single wire 120, and/or the plurality of circumferential segments 130. In some embodiments, the polymeric covering 160 may be fixedly attached to the body portion 110, the single wire 120, and/or the plurality of circumferential segments 130.

In some embodiments, the polymeric covering 160 may extend along an inner surface of the endoprosthesis 100, the body portion 110, the single wire 120, and/or the plurality of circumferential segments 130. In some embodiments, the polymeric covering 160 may extend along an outer surface of the endoprosthesis 100, the body portion 110, the single wire 120, and/or the plurality of circumferential segments 130. In at least some embodiments, the body portion 110, the single wire 120, and/or the plurality of circumferential segments 130 may be embedded within the polymeric covering 160. In some embodiments, the anti-migration element 150, the plurality of anti-migration elements, etc. may be embedded within the polymeric covering 160. Other configurations, including combinations thereof, are also contemplated. Some suitable but non-limiting examples of polymeric materials for the polymeric covering 160 are discussed below.

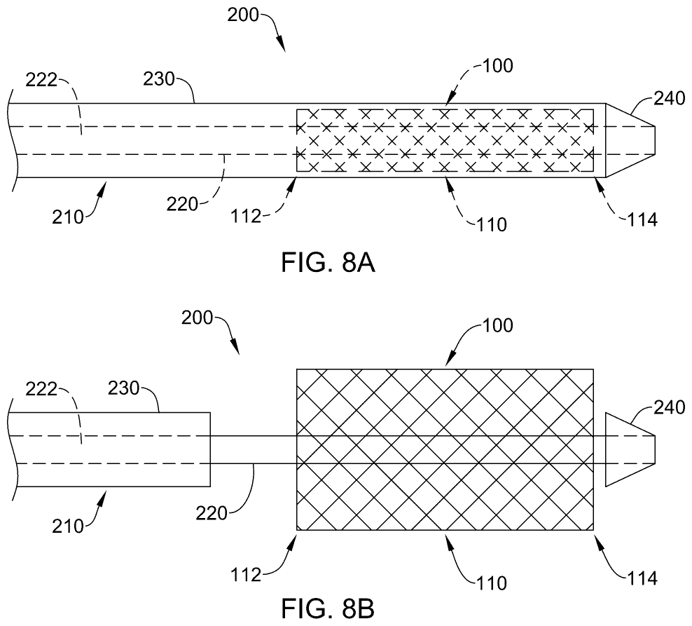

FIGS. 8A-8B illustrate selected aspects of an endoprosthesis system 200 comprising the endoprosthesis 100 and a delivery device 210. In some embodiments, the delivery device 210 may comprise an inner tubular member 220 and an outer tubular member 230. The endoprosthesis 100 may be disposable within an annular space disposed between the inner tubular member 220 and the outer tubular member 230 (e.g., within a lumen of the outer tubular member 230 and around and/or radially outward of the inner tubular member 220) in a collapsed delivery configuration. The inner tubular member 220 and the outer tubular member 230 may be axially movable relative to each other to load and/or deploy the endoprosthesis 100. In some embodiments, the inner tubular member 220 may comprise a guidewire lumen 222 extending therein. In some embodiments, the delivery device 210 may comprise a distal tip 240 disposed at a distalmost end of the inner tubular member 220. In at least some embodiments, the guidewire lumen 222 may extend through the distal tip 240. In alternative some embodiments, the delivery device 210 may comprise a crochet delivery device. Other configurations are also contemplated.

In some embodiments, the endoprosthesis 100 may be configured to shift between the collapsed delivery configuration, as seen in FIG. 8A, and an expanded deployed configuration, as seen in FIG. 8B. In some embodiments, the endoprosthesis 100 may be configured to self-expand from the collapsed delivery configuration to the expanded deployed configuration. In some embodiments, the endoprosthesis 100 may be balloon expandable from the collapsed delivery configuration to the expanded deployed configuration. Other configurations are also contemplated.

In some embodiments, the body portion 110 and/or the endoprosthesis 100 may be configured to minimize and/or avoid foreshortening and/or a change in the length of the body portion 110 and/or the endoprosthesis 100 as the body portion 110 and/or the endoprosthesis 100 shifts between the collapsed delivery configuration and the expanded deployed configuration. In some embodiments, the length of the body portion 110 and/or the endoprosthesis 100 from the first end 112 to the second end 114 may be configured to change by less than 10% when shifting between the collapsed delivery configuration and the expanded deployed configuration. In some embodiments, the length of the body portion 110 and/or the endoprosthesis 100 from the first end 112 to the second end 114 may be configured to change by less than 7.5% when shifting between the collapsed delivery configuration and the expanded deployed configuration. In some embodiments, the length of the body portion 110 and/or the endoprosthesis 100 from the first end 112 to the second end 114 may be configured to change by less than 5% when shifting between the collapsed delivery configuration and the expanded deployed configuration. In some embodiments, the length of the body portion 110 and/or the endoprosthesis 100 from the first end 112 to the second end 114 may be configured to change by less than 2.5% when shifting between the collapsed delivery configuration and the expanded deployed configuration. In some embodiments, the length of the body portion 110 and/or the endoprosthesis 100 from the first end 112 to the second end 114 may be configured to change by less than 1% when shifting between the collapsed delivery configuration and the expanded deployed configuration. Other configurations are also contemplated.

The materials that can be used for the various components of the endoprosthesis and/or the endoprosthesis system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery device, the endoprosthesis, the single wire, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester clastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylenc sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis, comprising:
a body portion extending axially from a first end to a second end along a central longitudinal axis;
wherein the body portion is formed from a single wire extending from the first end to the second end;
wherein the single wire forms a plurality of circumferential segments extending non-helically around the central longitudinal axis.

2. The endoprosthesis of claim 1, wherein each circumferential segment of the plurality of circumferential segments comprises an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion.

3. The endoprosthesis of claim 2, wherein each circumferential segment of the plurality of circumferential segments is connected to an axially adjacent circumferential segment of the plurality of circumferential segments by a transition segment of the single wire disposed at a third angle relative to the central longitudinal axis in the side view of the body portion, wherein the third angle is different from the first angle and the second angle.

4. The endoprosthesis of claim 3, wherein all transition segments of the single wire collectively define a transition zone.

5. The endoprosthesis of claim 4, wherein the transition zone extends parallel to the central longitudinal axis.

6. The endoprosthesis of claim 4, wherein the transition zone extends helically around the central longitudinal axis.

7. The endoprosthesis of claim 1, further comprising a polymeric covering fixedly attached to the body portion.

8. The endoprosthesis of claim 7, wherein the body portion is embedded within the polymeric covering.

9. The endoprosthesis of claim 1, wherein at least one circumferential segment of the plurality of circumferential segments comprises an anti-migration element projecting radially outward from the at least one circumferential segment, wherein the anti-migration element is formed from the single wire.

10. An endoprosthesis, comprising:
a body portion extending axially from a first end to a second end along a central longitudinal axis;
wherein the body portion is formed from a single wire extending continuously from the first end to the second end;
wherein the single wire forms a plurality of circumferential segments extending non-helically around the central longitudinal axis;
wherein the plurality of circumferential segments comprises a first end circumferential segment forming the first end, a second end circumferential segment forming the second end, and at least one medial circumferential segment disposed axially between the first end circumferential segment and the second end circumferential segment.

11. The endoprosthesis of claim 10, wherein each circumferential segment of the plurality of circumferential segments comprises an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion.

12. The endoprosthesis of claim 11, wherein each circumferential segment of the plurality of circumferential segments is connected to an axially adjacent circumferential segment by a transition segment of the single wire disposed nonparallel to the central longitudinal axis, the first struts, and the second struts in the side view of the body portion.

13. The endoprosthesis of claim 11, wherein the peaks and valleys of each circumferential segment define a circumferential segment height measured parallel to the central longitudinal axis, and the circumferential segment height is uniform among the plurality of circumferential segments.

14. The endoprosthesis of claim 11, wherein the peaks and valleys of each circumferential segment define a circumferential segment height measured parallel to the central longitudinal axis, and the circumferential segment height varies among the plurality of circumferential segments.

15. The endoprosthesis of claim 10, wherein a length of the body portion from the first end to the second end is configured to change by less than 5% when shifting between a collapsed delivery configuration and an expanded deployed configuration.

16. The endoprosthesis of claim 10, wherein each adjacent pair of circumferential segments defines an axial spacing, and the axial spacing between adjacent pairs of circumferential segments is uniform along the body portion.

17. The endoprosthesis of claim 10, wherein each adjacent pair of circumferential segments defines an axial spacing, and the axial spacing between adjacent pairs of circumferential segments varies along the body portion.

18. An endoprosthesis, comprising:
a body portion extending axially from a first end to a second end along a central longitudinal axis;
wherein the body portion is formed from a single wire extending from the first end to the second end;
wherein the single wire forms a plurality of circumferential segments extending non-helically around the central longitudinal axis;
wherein each circumferential segment of the plurality of circumferential segments comprises an undulating arrangement of first struts and second struts defining peaks and valleys, the first struts being disposed at a first angle relative to the central longitudinal axis in a side view of the body portion and the second struts being disposed at a second angle relative to the central longitudinal axis in the side view of the body portion;
wherein the peaks and valleys of adjacent circumferential segments of the plurality of circumferential segments are axially aligned with each other parallel to the central longitudinal axis.

19. The endoprosthesis of claim 18, wherein each circumferential segment of the plurality of circumferential segments is connected to an axially adjacent circumferential segment of the plurality of circumferential segments by a transition segment of the single wire;
wherein the transition segment extends between a valley of one circumferential segment and a peak of an immediately adjacent circumferential segment.

20. The endoprosthesis of claim 19, wherein the transition segment is nonparallel with the first struts and second struts of the one circumferential segment and the first struts and second struts of the immediately adjacent circumferential segment in the side view of the body portion.
